(19) United States
(12) Patent Application Publication
(10) Pub. No.: US 2026/0092036 A1
(43) Pub. Date: Apr. 2, 2026

(54) CRYSTALLINE SALTS OF A T-TYPE CALCIUM CHANNEL MODULATOR AND METHODS OF USE THEREOF

(71) Applicant: Praxis Precision Medicines, Inc., Boston, MA (US)

(72) Inventors: Daniel Thomas Walters, Jamesburg, NJ (US); Thomas San Ie, Evanston, IL (US)

(21) Appl. No.: 19/311,030

(22) Filed: Aug. 27, 2025

Related U.S. Application Data
(63) Continuation of application No. PCT/US2024/017962, filed on Mar. 1, 2024.
(60) Provisional application No. 63/449,461, filed on Mar. 2, 2023.

(51) Int. Cl. C07D 211/26; A61K 31/445; A61K 47/38

(52) U.S. Cl. CPC C07D 211/26; A61K 31/445; A61K 47/38

(57) ABSTRACT

The present disclosure provides Pattern D, i.e., a crystalline form of N-((1-(2-(tert-butylamino)-2-oxoethyl)piperidin-4-yl)methyl)-3-chloro-5-fluorobenzamide hydrochloride salt trihydrate (Compound 1). The present disclosure also provides pharmaceutical compositions comprising Compound 1 in the form of Pattern D, as well as methods of treating neurological and psychiatric disorders by administering to a subject in need thereof Compound 1 in the form of Pattern D or a pharmaceutical composition comprising Compound 1 in the form of Pattern D.

### RELATED APPLICATIONS

This application is a continuation of International Patent Application No. PCT/US2024/017962, filed on Mar. 1, 2024; which claims priority to U.S. Provisional Patent Application No. 63/449,461, filed on Mar. 2, 2023. The entire contents of each of the foregoing applications are hereby incorporated herein by reference.

### INTRODUCTION

T-type calcium channels are low-voltage activated ion channels that mediate the influx of calcium into cells. Aberrant function of these ion channels is associated with several diseases or conditions, including psychiatric disorder (e.g., mood disorder (e.g., major depressive disorder)), pain, tremor (e.g., essential tremor), epilepsy, or an epilepsy syndrome (e.g., absence seizures and juvenile myoclonic epilepsy). Accordingly, compounds that selectively modulate T-type calcium channel in mammals may be useful in treatment of such disease states.

### SUMMARY OF THE INVENTION

Accordingly, in some aspects, the present disclosure provides a crystalline form of N-((1-(2-(tert-butylamino)-2-oxoethyl)piperidin-4-yl)methyl)-3-chloro-5-fluorobenzamide hydrochloride salt trihydrate (Compound 1) characterized by an x-ray powder diffraction (XRPD) pattern comprising at least one peak at the diffraction angle (° 2θ) selected from the group consisting of:
a peak at approximately 12.0°;
a peak at approximately 15.6°;
a peak at approximately 16.7°;
a peak at approximately 19.8°;
a peak at approximately 21.2°;
a peak at approximately 24.1°;
a peak at approximately 25.2°;
a peak at approximately 27.3°; and
a peak at approximately 30.2°.

In some embodiments, the crystalline form is characterized by an XRPD pattern comprising the following peaks at the diffraction angle (° 2θ):
a peak at approximately 25.2°; and
a peak at approximately 27.3°.

In some embodiments, the crystalline form is characterized by an XRPD pattern comprising the following peaks at the diffraction angle (° 2θ):
a peak at approximately 24.1°;
a peak at approximately 25.2°; and
a peak at approximately 27.3°.

In some embodiments, the crystalline form is characterized by an XRPD pattern comprising the following peaks at the diffraction angle (° 2θ):
a peak at approximately 16.7°;
a peak at approximately 24.1°;
a peak at approximately 25.2°; and
a peak at approximately 27.3°.

In some embodiments, the crystalline form is characterized by an XRPD pattern comprising the following peaks at the diffraction angle (° 2θ):
a peak at approximately 12.0°;
a peak at approximately 16.7°;
a peak at approximately 24.1°;
a peak at approximately 25.2°; and
a peak at approximately 27.3°.

In some embodiments, the crystalline form is characterized by an XRPD pattern substantially the same as the XRPD pattern depicted in FIG. 2.

In some embodiments, the crystalline form has a melting point onset as determined by differential scanning calorimetry (DSC) at about 75.2° C.

In some embodiments, the crystalline form has a crystal structure characterized as a space group P1̅. In some embodiments, the crystal structure is characterized by an asymmetric unit cell with a volume of 1213.0(10) Å³ and 3-D parameters of a=8.584(3) Å; b=9.11(4) Å; c=17.043 Å.

In some embodiments, the present disclosure provides a pharmaceutical composition comprising the crystalline form of the present disclosure and a pharmaceutically acceptable carrier.

In some embodiments, the pharmaceutical composition further comprises a modified release-polymer. In some embodiments, the modified-release polymer is selected from the group consisting of a hydrophilic matrix polymer, a hydrophobic matrix polymer and a polyacrylate polymer. In some embodiments, the hydrophilic matrix polymer is hypromellose.

In some embodiments, the pharmaceutical composition is for oral administration.

In some embodiments, the present disclosure provides a method of treating a neurological disorder that comprises administering to a subject in need thereof the crystalline form of the present disclosure or the pharmaceutical composition of the present disclosure.

In some embodiments, the neurological disorder is tremor. In some embodiments, the tremor is essential tremor, Parkinson's tremor, cerebellar tremor or CACNA1G tremor. In some embodiments, the tremor is essential tremor.

In some embodiments, the present disclosure provides a method of treating a psychiatric disorder in a subject in need thereof, said method comprising administering to said subject the crystalline form of the present disclosure or the pharmaceutical composition of the present disclosure.

### BRIEF DESCRIPTION OF THE FIGURES

FIG. 1 is an X-ray powder diffraction (XRPD) spectrum of water activity experiments comparing Pattern C and Pattern D.

FIG. 2 is an XRPD spectrum of the new crystalline form of Compound 1, i.e., Pattern D.

FIG. 3 is a polarized light microscopy (PLM) image of Pattern D.

FIG. 4 shows the results of a thermo-gravimetric analysis (TGA) and differential scanning calorimetry (DSC) analysis of the new crystalline form of Pattern D.

FIG. 5 is an overlay of XRPD spectra of Pattern D heated to 90° C. and cooled back to room temperature; Pattern D heated to 150° C. and cooled back to room temperature; as well as Pattern C and Pattern D reference spectra.

FIG. 6 is an image of Pattern D heated to heated to 90° C. and 150° C. obtained using hot stage microscopy (HSM).

FIG. 7 is a Dynamic Vapor Sorption (DVS) spectrum collected on Pattern D.

FIG. 8 is an XRPD spectrum taken on Pattern D post DVS.

FIG. 9 is an overlay of XRPD spectra of a 2-week slurry of Pattern D in H₂O and of Pattern D reference.

FIG. 10 is a Thermal Ellipsoid plot of the compound in the crystal of Pattern D.

FIG. 11 shows hydrogen bonding network in Pattern D formed by the water and chloride anions.

FIG. 12 shows distances (Angstroms, red) and Angles (degrees, blue) in the solvent-anion network.

FIG. 13 shows a unit cell of Compound 1, with hydrogen atoms omitted for clarity.

FIG. 14 shows packing of Compound 1 as viewed along the b-axis.

FIG. 15 shows hydrogen bonding between molecules of Compound 1, with hydrogen atoms omitted for clarity.

### DETAILED DESCRIPTION OF THE INVENTION

### Crystalline Form of Compound 1 (Pattern D)

In some aspects, the present disclosure provides a crystalline form of N-((1-(2-(tert-butylamino)-2-oxoethyl)piperidin-4-yl)methyl)-3-chloro-5-fluorobenzamide hydrochloride salt trihydrate (Compound 1). In some embodiments, Compound 1 is represented by the following structure:

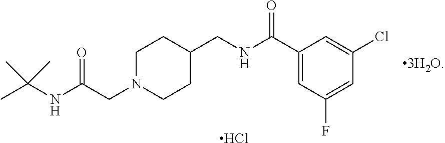 [SMILES: CC(C)(C)NC(=O)CN1CCC(CNC(=O)c2cc(F)cc(Cl)c2)CC1 ; Cl]

Compound 1 is a novel selective T-type calcium channel blocker designed to block abnormal neuron burst firing in the Cerebello-Thalamo-Cortical (CTC) circuit correlated with tremor activity. Certain crystalline forms of Compound 1 are known and described in, e.g., WO/2021/007487, the entire contents of which are hereby incorporated herein by reference.

The present disclosure provides novel crystalline forms of Compound 1. The terms “crystalline” or “crystalline form”, as used herein, refer to a solid form of Compound 1 in which atoms are arranged in regular, repeating patterns. In some embodiments, the term “crystalline” encompasses a polymorphic form or a non-amorphous form of Compound 1, without distinction.

The terms “amorphous” or “amorphous form”, as used herein, refer to a non-crystalline form of a substance, e.g., solid forms without a regular atomic arrangement.

The terms “polymorph” or “polymorphic form”, as used herein, refer to an organized structure involving only molecules of the solute and having a characteristic crystalline signature. These terms may refer to different crystalline forms of the same molecule. Different polymorphs may have different physical properties such as, for example, melting temperature, heat of fusion, solubility, dissolution rate and/or vibrational spectra as a result of different arrangements or conformations of the molecules in the crystal lattice. The differences in physical properties exhibited by different polymorphs may affect parameters important for pharmaceutical substances, such as storage stability, compressibility and density (important in formulation and product manufacturing), and dissolution rate (an important factor in bioavailability).

Differences in stabilities of different polymorphs may also result from differences in chemical reactivity (e.g., different susceptibility to oxidation). Thus, a dosage form comprised of one polymorph may discolor more rapidly than a dosage form comprised of a different polymorph of the same substance. Differences in stabilities of different polymorphs may also result from differences in mechanical properties (e.g., tablets may crumble on storage as a kinetically favored polymorph converts to a thermodynamically more stable polymorph); or from differences in both chemical and mechanical properties (e.g., tablets of one polymorph are more susceptible to breakdown at high humidity). As a result of solubility/dissolution differences, in extreme cases, some polymorphic transitions may result in loss of potency or, at the other extreme, toxicity. In addition, physical properties of the crystal may be important in processing. For example, different polymorphs of the same substances may exhibit differences in their propensity to form solvates or in their particle shape and size distributions, affecting purification (e.g., one polymorph may be more difficult to filter and wash free of impurities than another polymorph).

Polymorphs of a molecule may be obtained by a number of methods, as known in the art. Such methods may include, but are not limited to, melt recrystallization, melt cooling, solvent recrystallization, desolvation, rapid evaporation, rapid cooling, slow cooling, vapor diffusion and sublimation. Techniques for characterizing polymorphs may include, but are not limited to, differential scanning calorimetry (DSC), X-ray powder diffractometry (XRPD), single crystal X-ray diffractometry, vibrational spectroscopy, e.g., IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies. Specifically, XRPD is a technique used to characterize the crystallographic structure, size, and preferred orientation in polycrystalline or powdered solid samples. This diffraction is also used to characterize heterogeneous solid mixtures to determine the percent of crystalline compounds present and can provide structural information on unknown materials. The term “X-ray powder diffraction pattern, used herein interchangeably with the term “XRPD pattern” refers to a graphical representation of the data collected by XRPD analysis.

In some embodiments, the present disclosure provides a new crystalline form of Compound 1, which is also referred to herein as “Pattern D”.

In some embodiments, the new crystalline form of Compound 1, i.e., Pattern D, is characterized by an X-ray powder diffraction pattern (XRPD pattern) that includes at least one peak, e.g., at least two peaks, at least three peaks, at least four peaks, at least five peaks, at least six peaks, at least seven peaks, at least eight peaks or nine peaks, at the diffraction angle (° 2θ) selected from the group consisting of:
a peak at approximately 12.0°;
a peak at approximately 15.6°;
a peak at approximately 16.7°;
a peak at approximately 19.8°;
a peak at approximately 21.2°;
a peak at approximately 24.1°;
a peak at approximately 25.2°;
a peak at approximately 27.3°; and
a peak at approximately 30.2°.

The term “peak”, as used herein, refers to a peak in the XRPD pattern having an intensity at least 20%, e.g., at least 30%, at least 40%, at least 50% or at least 100% greater than the baseline noise.

The terms “approximately” or “about”, as used herein in reference to a peak in an XRPD pattern, refer to the XRPD pattern in which the peak appears within 0.5° 2θ, e.g., within 0.4, 0.3, 0.2, 0.1, 0.05 or 0.01 °2θ of a given ° 2θ value.

In some embodiments, the new crystalline form of Compound 1, i.e., Pattern D, is characterized by an X-ray powder diffraction pattern (XRPD pattern) that includes at least one peak, e.g., at least two peaks, at least three peaks, at least four peaks, at least five peaks, at least six peaks, at least seven peaks, at least eight peaks or nine peaks, at the diffraction angle (° 2θ) selected from the group consisting of:
a peak at 12.0°±0.2°;
a peak at 15.6°±0.2°;
a peak at 16.7°±0.2°;
a peak at 19.8°±0.2°;
a peak at 21.2°±0.2°;
a peak at 24.1°±0.2°;
a peak at 25.2°±0.2°;
a peak at 27.3°±0.2°; and
a peak at 30.2°±0.2°.

In some embodiments, the present disclosure provides a new crystalline form of Compound 1, i.e., Pattern D, that is characterized by an X-ray powder diffraction pattern (XRPD pattern) that includes at least two peaks selected from the group of peaks listed above.

In some embodiments, the present disclosure provides a new crystalline form of Compound 1, i.e., Pattern D, that is characterized by an X-ray powder diffraction pattern (XRPD pattern) that includes at least three peaks selected from the group of peaks listed above.

In some embodiments, the present disclosure provides a new crystalline form of Compound 1, i.e., Pattern D, that is characterized by an X-ray powder diffraction pattern (XRPD pattern) that includes at least four peaks selected from the group of peaks listed above.

In some embodiments, the present disclosure provides a new crystalline form of Compound 1, i.e., Pattern D, that is characterized by an X-ray powder diffraction pattern (XRPD pattern) that includes at least five peaks selected from the group of peaks listed above.

In some embodiments, the present disclosure provides a new crystalline form of Compound 1, i.e., Pattern D, that is characterized by an X-ray powder diffraction pattern (XRPD pattern) that includes at least six peaks selected from the group of peaks listed above.

In some embodiments, the present disclosure provides a new crystalline form of Compound 1, i.e., Pattern D, that is characterized by an X-ray powder diffraction pattern (XRPD pattern) that includes at least seven peaks selected from the group of peaks listed above.

In some embodiments, the present disclosure provides a new crystalline form of Compound 1, i.e., Pattern D, that is characterized by an X-ray powder diffraction pattern (XRPD pattern) that includes at least eight peaks selected from the group of peaks listed above.

In some embodiments, the present disclosure provides new crystalline form of Compound 1, i.e., Pattern D, that is characterized by an X-ray powder diffraction pattern (XRPD pattern) that includes all nine peaks from the group of peaks listed above.

In some embodiments, the new crystalline form of Compound 1, i.e., Pattern D, is characterized by an XRPD pattern comprising the following peaks at the diffraction angle (° 2θ):
a peak at approximately 25.2°; and
a peak at approximately 27.3°.

In some embodiments, the new crystalline form of Compound 1, i.e., Pattern D, is characterized by an XRPD pattern comprising the following peaks at the diffraction angle (° 2θ)
a peak at 25.2°±0.2°; and
a peak at 27.3°±0.2°.

In some embodiments, the new crystalline form of Compound 1, i.e., Pattern D, is characterized by an XRPD pattern comprising the following peaks at the diffraction angle (° 2θ) a peak at approximately 24.1°;
a peak at approximately 25.2°; and
a peak at approximately 27.3°.

In some embodiments, the new crystalline form of Compound 1, i.e., Pattern D, is characterized by an XRPD pattern comprising the following peaks at the diffraction angle (° 2θ) a peak at 24.1°±0.2°;
a peak at 25.2°±0.2°; and
a peak at 27.3°±0.2°.

In some embodiments, the new crystalline form of Compound 1, i.e., Pattern D, is characterized by an XRPD pattern comprising the following peaks at the diffraction angle (° 2θ):
a peak at approximately 16.7°;
a peak at approximately 24.1°;
a peak at approximately 25.2°; and
a peak at approximately 27.3°.

In some embodiments, the new crystalline form of Compound 1, i.e., Pattern D, is characterized by an XRPD pattern comprising the following peaks at the diffraction angle (° 2θ):
a peak at 16.7°±0.2°;
a peak at 24.1°±0.2°;
a peak at 25.2°±0.2°; and
a peak at 27.3°±0.2°.

In some embodiments, the new crystalline form of Compound 1, i.e., Pattern D, is characterized by an XRPD pattern comprising the following peaks at the diffraction angle (° 2θ) a peak at approximately 12.0°;
a peak at approximately 16.7°;
a peak at approximately 24.1°;
a peak at approximately 25.2°; and
a peak at approximately 27.3°.

In some embodiments, the new crystalline form of Compound 1, i.e., Pattern D, is characterized by an XRPD pattern comprising the following peaks at the diffraction angle (° 2θ):
a peak at 12.0°±0.2°;
a peak at 16.7°±0.2°;
a peak at 24.1°±0.2°;
a peak at 25.2°±0.2°; and
a peak at 27.3°±0.2°.

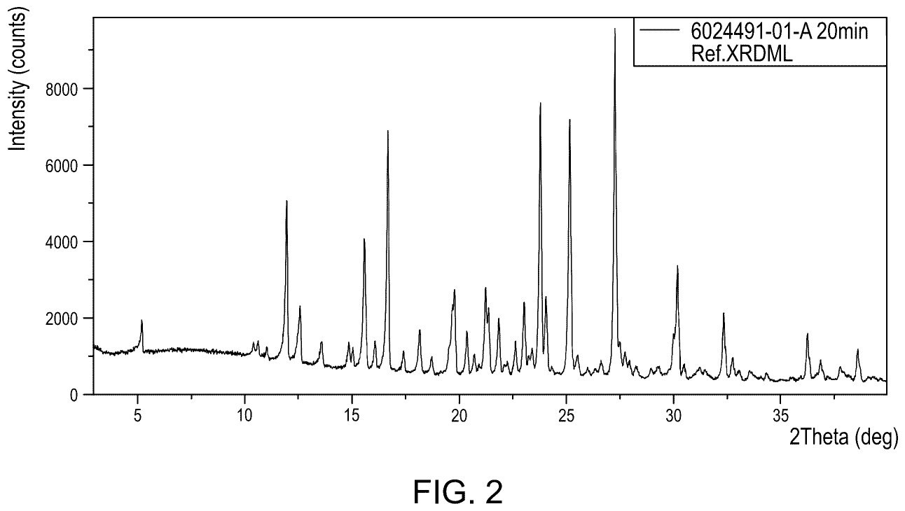

In some embodiments, the new crystalline form of Compound 1, i.e., Pattern D, is characterized by an XRPD pattern substantially the same as the XRPD pattern depicted in FIG. 2.

In some embodiments, the new crystalline form of Compound 1, i.e., Pattern D, has a melting point onset as determined by differential scanning calorimetry (DSC) at about 75.2° C.

In some embodiments, the new crystalline form of Compound 1, i.e., Pattern D, has a crystal structure characterized as a space group P1̅.

In some embodiments, the new crystalline form of Compound 1, i.e., Pattern D, is characterized by an asymmetric unit cell with a volume of 1213.0(10) Å³ and 3-D parameters of a=8.584(3) Å; b=9.11(4) Å; c=17.043 Å.

In some aspects, the new crystalline form of Compound 1, i.e., Pattern D, is at least 90% pure, as expressed by weight of the Pattern D vs. weight of a composition comprising Pattern D (w/w %). For example, the Pattern D may be at least 95% pure, at least 96% pure, at least 97% pure, at least 98% pure, at least 99% pure, at least 99.1% pure, at least 99.5% pure, or at least 99% pure. As used herein, the terms “pure” or “purity” refer to a compound that is about 90-100% pure, e.g., about 95-100% pure, 98-100% pure, or about 99-100% pure. In some embodiments, the Pattern D that is pure comprises less than about 10%, less than about 5%, less than about 2% or less than about 1% of impurities. The impurities may include, e.g., one or more of degradation products, oxidized products, solvents, and/or other undesirable impurities.

### Methods of Synthesizing Crystalline Form of Compound 1 (Pattern D)

The new crystalline form of Compound 1, i.e., Pattern D, provided by the present disclosure may be prepared by crystallizing an amorphous form of Compound 1 from a solvent system that is characterized by a water activity of greater than about 0.6, e.g., 0.7 or greater, 0.8 or greater, 0.9 or greater or 1.0. In some embodiments, Pattern D may be prepared by crystallizing an amorphous form of Compound 1 from a solvent system that is characterized by a water activity of 7.0 or greater. In some embodiments, Pattern D may be prepared by crystallizing an amorphous form of Compound 1 from a solvent system that is characterized by a water activity of 8.0 or greater.

The term “water activity (a_{w})”, as used herein, refers to the equilibrium amount of water available for hydration of materials. In some embodiments, the term “water activity (a_{w})” refers to the ratio of the vapor pressure of water in a solvent system to the vapor pressure of pure water at the same temperature. Water activity is a function of water concentration and the nature of the co-solvent in a solvent system, and may be measured by methods known in the art.

In some embodiments, the solvent system characterized by a water activity of greater than about 0.6, e.g., 0.7 or greater, 0.8 or greater, 0.9 or greater or 1.0, comprises water. In some embodiments, the solvent system characterized by a water activity of greater than about 0.6, e.g., 0.7 or greater, 0.8 or greater, 0.9 or greater or 1.0, comprises only water and does not comprise an organic solvent. In some embodiments, the solvent system characterized by a water activity of greater than about 0.6, e.g., 0.7 or greater, 0.8 or greater, 0.9 or greater or 1.0, comprises water and an organic solvent, e.g., acetone, acetonitrile, benzene, chloroform, cyclohexane, n-hexane, diethyl ether, dimethyl sulfoxide, dimethyl formamide, ethyl acetate, methyl t-butyl ether, tetrahydrofuran, or an alcohol (such as methanol, ethanol or isopropanol). In some embodiments, the solvent system comprises an water and an organic solvent, e.g., acetone, at a ratio of water:acetone of greater than 1:3.

In some embodiments, the present disclosure provides a new crystalline form of Compound 1, i.e., Pattern D, prepared by the methods described above.

### Compositions Comprising Crystalline Form of Compound 1 (Pattern D)

The present disclosure also provides pharmaceutical compositions comprising the new crystalline salt form of a Compound 1, i.e., Pattern D. The amount of Compound 1 in the form of Pattern D in a provided composition is such that Compound 1 can act as an effective T-type calcium channel blocker. For example, the amount of Compound 1 in the form of Pattern D in a pharmaceutical composition is such that it is effective for treating, e.g., a neurological disorder, a psychiatric disorder, a generalized epileptic syndrome, or an essential tremor.

Pharmaceutical compositions described herein can be prepared by any method known in the art of pharmacology. In general, such preparatory methods include the steps of bringing Compound 1 in the form of Pattern D into association with a carrier and/or one or more other accessory ingredients, and then, if necessary and/or desirable, shaping and/or packaging the product into a desired single- or multi-dose unit.

Pharmaceutically acceptable carriers used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Carriers such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, sodium stearyl fumarate, and mixtures thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

The pharmaceutical compositions provided herein may be administered orally, parenterally, by inhalation spray, topically, rectally, nasally, buccally, transmucosally, or in an ophthalmic preparation. The term “parenteral” as used herein includes subcutaneous, intravenous, intramuscular, intra-articular, intra-synovial, intrasternal, intrathecal, intrahepatic, intralesional and intracranial injection or infusion techniques. In one aspect, the pharmaceutical compositions provided herein are orally administered in an orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The amount of Compound 1 in the form of Pattern D to be combined with carrier materials to produce a composition in a single dosage form will vary depending upon the subject to be treated and the particular mode of administration. For example, a specific dosage and treatment regimen for any particular subject will depend upon a variety of factors, including age, body weight, general health, sex, diet, time of administration, rate of excretion, drug combination, the judgment of the treating physician, and the severity of the particular disease being treated. In one aspect, a pharmaceutical composition may be formulated such that a dosage equivalent to about 0.001 to about 100 mg/kg body weight/day of Compound 1 in the form of Pattern D (e.g., about 0.5 to about 100 mg/kg of Compound 1) can be administered to a subject receiving these compositions. Alternatively, dosages equivalent to 1 mg/kg and 1000 mg/kg of compound 1 every 4 to 120 hours are also acceptable. As used herein, the dose refers to the amount of Compound 1 in the form of Pattern D.

In some embodiments, Compound 1 in the form of Pattern D is formulated for administration at a dose equivalent to about 2 mg to about 3000 mg of Compound 1, e.g., about 5 mg to about 350 mg, about 5 mg to about 200 mg, about 5 mg to about 100 mg, about 20 mg to about 40 mg of Compound 1 in the form of Pattern D. In certain embodiments, the Compound of Formula 1 in the form of Pattern D is formulated for administration at a dose equivalent to about 5 mg, about 10 mg, about 15 mg, about 20 mg, about 30 mg, about 40 mg, about 45 mg, about 50 mg, about 60 mg, about 70 mg, about 80 mg, about 90 mg, about 100 mg, about 110 mg or about 120 mg of Compound 1 in the form of Pattern D.

In some embodiments, Compound 1 in the form of Pattern D is formulated for administration at a dose equivalent to about 2 mg to about 3000 mg of Compound 1 per day, e.g., about 5 mg to about 500 mg, about 5 mg to about 200 mg, about 20 mg to about 40 mg of Compound 1 per day. In some embodiments, Compound 1 in the form of Pattern D is formulated for administration at a dose equivalent to about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, or about 150 mg of Compound 1 per day. In some embodiments, Compound 1 in the form of Pattern D may be administered once per day. In other embodiments, Compound 1 in the form of Pattern D maybe administered twice per day. In yet other embodiments, Compound 1 in the form of Pattern D may be administered once every other day.

In some embodiments, Compound 1 in the form of Pattern D may be formulated as a tablet composition together with a pharmaceutically acceptable carrier. Suitable carriers that may be used in a tablet composition may be selected from one or more of microcrystalline cellulose, mannitol, Croscarmellose Sodium, and Sodium Stearyl Fumarate. For example, in some embodiments, the carrier may be microcrystalline cellulose, e.g., present in the tablet composition in an amount of about 40% w/w to about 80% w/w, e.g., about 50% w/w to about 70% w/w, or about 55% w/w to about 65% w/w. In some embodiments, the carrier may be mannitol, e.g., present in the tablet composition in an amount of 20% w/w to about 45% w/w, e.g., about 20% w/w to about 30% w/w. In some embodiments, the carrier may be croscarmellose sodium, e.g., present in an amount of about 1% w/w to about 5% w/w. In some embodiments, the carrier may be stearyl fumarate, e.g., present in in the tablet composition an amount of about 1% w/w to about 5% w/w.

In some embodiments, Compound 1 in the form of Pattern D may be present in the tablet composition in an amount equivalent to about 1 mg to about 150 mg of Compound 1, e.g., about 1 mg to about 100 mg, about 5 mg to about 50 mg or about 20 mg to about 40 mg of Compound 1.

In some embodiments, a pharmaceutical composition provided by the present disclosure may be administered to the subject once per day or more than once a day (e.g., twice a day, three times a day, or four times a day).

In some embodiments, methods of the present disclosure comprise administering to a subject in need thereof a titrated dose of Compound 1 in the form of Pattern D. In some embodiments, the maximum titrated dose is 60 mg per day or 100 mg per day. In one embodiment, the maximum titrated dose is 60 mg per day.

In one aspect, the pharmaceutical compositions provided by the present disclosure include a single-unit dosage form comprising the Compound 1 in the form of Pattern D. In certain embodiments, single-unit dosage form comprises up to 200 mg of the compound of Compound 1 in the form of Pattern D. In some embodiments, the single-unit dosage form comprises a length of up to 16 mm, e.g., about 14 mm to about 16 mm, and/or a width of up to 7 mm, e.g., about 5 mm to about 7 mm. In some embodiments, the single-unit dosage form is bioequivalent to a reference composition of the same dosage strength administered as multiple dosage forms, such as the a modified release (MR) formulation comprising the Compound 1 in the form of Pattern D available as 5 mg and 20 mg tablets that are round and small in size (e.g., about 6 mm in diameter). In some embodiments, bioequivalency may be established by: (a) a 90% Confidence Interval for AUC which is between about 80% and about 125%, and (b) a 90% Confidence Interval for Cₘₐₓ, which is between about 80% and about 125%.

### Modified-Release Dosage Forms and Compositions

In some embodiments, the present disclosure also provides a pharmaceutical composition comprising Compound 1 in the form of Pattern D and an excipient that functions to modify the release rate of the Compound 1. In some embodiments, the pharmaceutical composition may be a swellable core technology formulation.

A composition of the present disclosure may comprise Compound 1 in the form of Pattern D and a modified-release polymer. The term “modified-release polymer”, as used herein, refers to any polymer that, when added to a composition comprising Compound 1 in the form of Pattern D, modifies the release rate of Compound 1. A modified-released polymer may be a hydrophilic polymer, e.g., a hydroxypropyl methyl cellulose (HPMC) polymer; a hydrophobic polymer, e.g., ethyl cellulose or ethocell; or a polyacrylate polymer, e.g., Eudragit RL100, Eudragit RS100. In some embodiments, the modified-release polymer is present in the composition in an amount sufficient to modify the release rate of Compound 1. In some embodiments, the composition is for oral administration.

In some embodiments, a composition of the disclosure may comprise a modified-release polymer and Compound 1 in the form of Pattern D in an amount of from about 0.9% by weight to about 40% by weight (e.g., from about 0.9% by weight to about 30%, from about 1% by weight to about 25% by weight, from about 2% by weight to about 25% by weight, from about 3% by weight to about 20% by weight, from about 4% by weight to about 20% by weight, from about 5% by weight to about 20% by weight, from about 5% by weight to about 15% by weight, from about 5% by weight to about 10% by weight, or about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40% by weight.

In some embodiments, a composition of the disclosure may comprise a modified-release polymer and Compound 1 in the form of Pattern D in an amount of from about 1 mg to about 40 mg, e.g., about 5 mg, about 10 mg, about 15 mg, about 20 mg, about 25 mg, about 30 mg, about 35 mg, about 40 mg.

In some embodiments, the composition comprises from about 55 mg to about 65 mg of a modified-release polymer (e.g., an HPMC polymer). In some embodiments, the composition comprises from about 10% by weight to about 70% by weight, e.g., from about 50% by weight to about 60% by weight of the modified-release polymer (e.g., an HPMC polymer).

In some embodiments, the composition may further comprise a diluent, e.g., microcrystalline cellulose. In some embodiments, the composition may comprise microcrystalline cellulose in an amount from about 15 mg to 40 mg, e.g., from about 15 mg to about 25 mg, from about 20 mg to about 25 mg, from about 25 mg to about 30 mg, or from about 30 mg to about 40 mg. In some embodiments, the composition may comprise microcrystalline cellulose in an amount of from about 15% to about 35% by weight, e.g., from about 15% to about 20%, from about 20% to about 25%, from 25% to about 30%, or from 30% to about 35% by weight.

In some embodiments, the composition may further comprise a glidant, e.g., colloidal silicon dioxide. In some embodiments, the composition may further comprise a lubricant, e.g., magnesium stearate. In some embodiments, the composition may further comprise a coating.

In some embodiments, the composition may be administered to a patient once daily or twice daily. In some embodiments, the composition may be in a form of a tablet, a capsule or a suspension. In one embodiment, the composition is in a form of a tablet.

### Methods of Treatment Using Compound 1 in the Form of Pattern D

The present disclosure also provides methods of treating a disease, disorder or condition relating to function of T-type calcium channel. The method comprise administering to a subject in need thereof an effective amount of Compound 1 in the form of Pattern D described herein, or a pharmaceutical composition comprising Compound 1 in the form of Pattern D described herein. The ability of Compound 1 to modulate T-type Calcium Channels has been described, e.g., in at least, WO 2021/007487, the entire contents of which are hereby incorporated herein by reference.

In some embodiments, methods of the present disclosure comprising administering to a subject in need thereof a single-unit dosage form described herein.

In some embodiments, the present disclosure provides a method of treating a neurological disorder in a subject in need thereof, the method comprising administering to the subject an effective amount of Compound 1 in the form of Pattern D. In some embodiments, the subject has a mutation in one or both of the T-type calcium channel genes CACNA1H and CACNA1G. In some embodiments, the neurological disorder is epilepsy. In some embodiments, the epilepsy is juvenile epilepsy. In some embodiments, the epilepsy is genetic epilepsy. In some embodiments, the neurological disorder is absence seizure. In some embodiments, the neurological disorder is pain (e.g., acute pain, chronic pain, neuropathic pain, inflammatory pain, nociceptive pain, central pain, thalamic pain or migraine. In some embodiments, the neurological disorder is tremor (e.g., essential tremor, or a Parkinsonian tremor). In some embodiments, the neurological disorder is ataxia (e.g., spinocerebellar ataxia, or spinocerebellar ataxia with CACNA1G mutations). In some embodiments, the neurological disorder is tinnitus. In certain embodiments, the neurological disorder is a disorder of wakefulness.

In some embodiments, Compound 1 in the form of Pattern D, or a pharmaceutical composition comprising Compound 1 in the form of Pattern D described herein, is effective in the treatment of tremor (e.g., essential tremor). In some embodiments, Compound 1 in the form of Pattern D, or a pharmaceutical composition comprising Compound 1 in the form of Pattern D described herein, is effective in the treatment of an epilepsy or epilepsy syndrome, e.g., absence seizures, juvenile myoclonic epilepsy, status epilepticus, or a genetic epilepsy. Compound 1 in the form of Pattern D, or a pharmaceutical composition comprising Compound 1 in the form of Pattern D described herein may also modulate all T-type calcium channels, e.g., Cav3.1, Cav3.2, and/or Cav3.3. In some embodiments, Compound 1 in the form of Pattern D, or a pharmaceutical composition comprising Compound 1 in the form of Pattern D described herein, effective in the treatment of a psychiatric disorder, e.g., mood disorder, e.g., major depressive disorder.

In some embodiments, the present disclosure also provides a method of treating a psychiatric disorder in a subject in need thereof, the method comprising administering to the subject an effective amount of Compound 1 in the form of Pattern D or a pharmaceutical composition comprising Compound 1 in the form of Pattern D described herein. In some embodiments, the psychiatric disorder is a mood disorder. In some embodiments, the mood disorder is major depressive disorder.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human” and “patient” may be used interchangeably herein.

### Epilepsy and Epilepsy Syndromes

Compound 1 in the form of Pattern D or a pharmaceutical composition comprising Compound 1 in the form of Pattern D described herein may be useful in the treatment of epilepsy and epilepsy syndromes. Epilepsy is a CNS disorder in which nerve cell activity in the brain becomes disrupted, causing seizures which can manifest as abnormal movements, periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure or a partial or focal seizure. All areas of the brain are involved in a generalized seizure. A person experiencing a generalized seizure may cry out or make some sound, stiffen for several seconds to a minute and then have rhythmic movements of the arms and legs. The eyes are generally open, the person may appear not to be breathing and actually turn blue. The return to consciousness is gradual and the person maybe confused from minutes to hours. The following are the main types of generalized seizures: tonic-clonic, tonic, clonic, myoclonic, myoclonic-tonic-clonic, myoclonic-atonic, atonic, and absence (typical, atypical, myoclonic, eyelid myoclonia) seizures, and epileptic spasms. In a partial or focal seizure, only part of the brain is involved, so only part of the body is affected. Depending on the part of the brain having abnormal electrical activity, symptoms may vary.

Epilepsy, as described herein, includes a generalized, partial, complex partial (e.g., seizures involving only part of the brain, but where consciousness is compromised), tonic clonic, clonic, tonic, refractory seizures, status epilepticus, absence seizures, febrile seizures, or temporal lobe epilepsy.

Compound 1 in the form of Pattern D or a pharmaceutical composition comprising Compound 1 in the form of Pattern D described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises epileptic encephalopathy, Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency. In some embodiments, the epilepsy syndrome is childhood absence epilepsy (CAE). In some embodiments, the epilepsy syndrome is juvenile absence epilepsy (JAE). In some embodiments, the epilepsy syndrome is Lennox-Gastaut syndrome. In some embodiments, the epilepsy syndrome is SLC6A1 epileptic encephalopathy. In some embodiments, the epilepsy syndrome is associated with mutations in the genes that code for T-type calcium channels (e.g., CACNA1G, EEF1A2, and GABRG2 for genetic generalized epilepsy (GGE) and LGI1, TRIM3, and GABRG2 for non-acquired focal epilepsy (NAFE)). In some embodiments, the epilepsy syndrome is Doose syndrome or myoclonic astatic epilepsy. In some embodiments, the epilepsy syndrome is epileptic encephalopathy with continuous spike and wave during sleep (CSWS). In some embodiments, the epilepsy syndrome is Landau Kleffner Syndrome (LKS). In some embodiments, the epilepsy syndrome is Jeavons syndrome.

### Absence Seizures

Absence seizures are one of the most common seizure types in patients with idiopathic generalised epilepsy (IGE). Absence seizures are relatively brief, non-convulsive seizures characterised by abrupt onset of loss of awareness and responsiveness, usually lasting between 10-30 seconds in duration, with a rapid return to normal consciousness without post-ictal confusion. The seizures are characterised on an accompanying EEG recording by the abrupt onset and offset of generalised 1-6 Hz (e.g., 3 Hz) spike and wave discharges. Absence seizure often occur multiple times per day, interrupt learning and psychosocial functioning, and present a risk of injury because of the frequent episodes of loss of awareness. Typically, absence seizures begin in early childhood and remit by teenage years. However, in a minority of patients they persist into adulthood where they are often drug resistant, and may be accompanied by other seizure types such as generalised tonic-clonic seizures. In these adult patients, the absence seizures are usually highly disabling, in particular by disqualifying the sufferer from obtaining a motor vehicle license or pursuing occupations and hobbies in which the seizures-associated periods of loss of awareness pose a safety risk, and are associated with significant psychosocial disabilities.

While there is a common perception that absence seizures are relatively “easy” to treat, a randomised control trial in patients with childhood absence epilepsy showed that even the most effective anti-epileptic drugs, ethosuximide and valproate, only completely controlled the seizures in 53% and 58% of patients respectively at 16 weeks as assessed by video-EEG recordings, and 45% and 44% respectively at 12 months. Lamotrigine, the other AED commonly used to treat absence seizures, only controlled the seizures in 29% of patients at 16 weeks, and 21% of patients at 12 months. Furthermore, both ethosuximide and valproate are commonly associated with intolerable side effects (occurring in 24% of patients treated with either of these drugs), and the latter is now generally considered to be contraindicated in girls and women of childbearing potential. Other treatment options for absence seizures are limited, with only benzodiazepines having established efficacy- and these are commonly poorly tolerated due to sedative and cognitive side effects. Absence seizures persisting into adult life are particularly difficult to treat, with patients often being treated with multiple drugs resulting in significant side-effects without attaining seizure control.

There is abundant evidence that low threshold (T-type) calcium channels play a critical role in the generation and maintenance of absence seizures, being a key component of the oscillatory burst firing that occurs in thalamocortical neurones during absence seizures. In some embodiments, the present disclosure provides a method of treating absence seizures in a subject in need thereof, the method comprising administering to the subject an effective amount of Compound 1 in the form of Pattern D or a pharmaceutical composition comprising Compound 1 in the form of Pattern D described herein. In some embodiments, the absence seizures are refractory absence seizures. In some embodiments, the absence seizures are refractory to an anti-epileptic drug (e.g., ethosuximide, valproic acid, or lamotrigine).

In some embodiments, the subject has epilepsy. In some embodiments, the absence seizures are atypical absence seizures. In some embodiments, the absence seizures comprise adult absence seizures, juvenile absence seizures, or childhood absence seizures.

In some embodiments, the methods described herein further comprise identifying a subject having absence seizures.

In some embodiments, the present disclosure provides a method of treating a generalized epileptic syndrome with absence seizures in a subject in need thereof, the method comprising administering to the subject an effective amount of Compound 1 in the form of Pattern D or a pharmaceutical composition comprising Compound 1 in the form of Pattern D described herein. In some embodiments, the method results in reduction of the number of seizures.

In some embodiments, the present disclosure provides a method of treating a generalized epileptic syndrome with absence seizures in a subject in need thereof, the method comprising administering to the subject an effective amount of Compound 1 in the form of Pattern D or a pharmaceutical composition comprising Compound 1 in the form of Pattern D described herein.

In some embodiments, the method of treating a generalized epileptic syndrome with absence seizures provided by the present disclosure results in reduction of the mean or total seizure duration.

In some embodiments, the method of treating a generalized epileptic syndrome with absence seizures provided by the present disclosure results in reduction of the seizure frequency, duration or both as measured by Electroencephalogram (EEG).

In some embodiments, the method of treating a generalized epileptic syndrome with absence seizures provided by the present disclosure results in a reduction of the mean seizure duration as measured by EEG.

In some embodiments, the method of treating a generalized epileptic syndrome with absence seizures provided by the present disclosure results in a reduction of the cumulative seizure duration as measured by EEG.

In some embodiments, the method of treating a generalized epileptic syndrome with absence seizures provided by the present disclosure results in a reduction of the total time with 2.5-4 Hz spike wave discharges after hyperventilation and photic stimulation challenges as measured by EEG.

In some embodiments, the method of treating a generalized epileptic syndrome with absence seizures provided by the present disclosure results in a reduction of global severity as measured by Clinical Global Impression-Severity (CGI-S) or Clinical Global Impression-Improvement (CGI-I) scores. CGI-S is a 7-point scale test to rate the severity of the patient's illness at the time of assessment, relative to the clinician's past experience with patients with the same diagnosis. CGI-I is a 7-point scale test to evaluate the improvement of the patient's illness relative to the baseline.

In some embodiments, the method of treating a generalized epileptic syndrome with absence seizures provided by the present disclosure results in a reduction of the number of seizures.

In some embodiments, the method of treating a generalized epileptic syndrome with absence seizures provided by the present disclosure results in a reduction of the seizure density as measured by Electroencephalogram (EEG).

In some embodiments, the method of treating a generalized epileptic syndrome with absence seizures provided by the present disclosure results in a reduction of the mean seizure duration as measured by EEG.

### Genetic Epilepsies

In some embodiments, the present disclosure provides a method of treating epilepsy or an epilepsy syndrome in a subject in need thereof, the method comprising administering to the subject an effective amount of Compound 1 in the form of Pattern D or a pharmaceutical composition comprising Compound 1 in the form of Pattern D described herein.

In some embodiments, the epilepsy or epilepsy syndrome is a genetic epilepsy or a genetic epilepsy syndrome. In some embodiments, the epilepsy or epilepsy syndrome is genetic generalized epilepsy. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) prior to administration of Compound 1 in the form of Pattern D or a pharmaceutical composition comprising Compound 1 in the form of Pattern D described herein.

In one aspect, the present disclosure provides a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) comprising administering to a subject in need thereof an effective amount of Compound 1 in the form of Pattern D or a pharmaceutical composition comprising Compound 1 in the form of Pattern D described herein.

Compound 1 in the form of Pattern D or a pharmaceutical composition comprising Compound 1 in the form of Pattern D described herein may also be used to treat an epileptic encephalopathy in a subject in need thereof, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, WWOX, CACNA1G, CACNA1H, and CACNA1I prior to administration of Compound 1 in the form of Pattern D or a pharmaceutical composition comprising Compound 1 in the form of Pattern D described herein.

Compound 1 in the form of Pattern D or a pharmaceutical composition comprising Compound 1 in the form of Pattern D described herein may also be used to treat an epileptic encephalopathy in a subject in need thereof, wherein the subject has a mutation in one or more of ADSL, ALDH5A1, ALDH7A1, ALG13, ARGI, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBL1XR1, AMT, GCSH, GLDC, FLNA, PTEN, and RANBP2.

In some embodiments, the methods described herein further comprise identifying a subject having a mutation in one or more of ADSL, ALDH5A1, ALDH7A1, ALG13, ARGI, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBL1XR1, AMT, GCSH, GLDC, FLNA, PTEN, and RANBP2 prior to administration of Compound 1 in the form of Pattern D or a pharmaceutical composition comprising Compound 1 in the form of Pattern D described herein.

Compound 1 in the form of Pattern D or a pharmaceutical composition comprising Compound 1 in the form of Pattern D described herein may also be used to treat an epileptic encephalopathy in a subject in need thereof, wherein the subject has a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBL1XR1, TCF4, TPP1 (CLN2), TSC1, TSC2, UBE3A, WDR45, WWOX, and ZEB2.

In some embodiments, the methods described herein further comprise identifying a subject having a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBL1XR1, TCF4, TPP1 (CLN2), TSC1, TSC2, UBE3A, WDR45, WWOX, and ZEB2 prior to administration of Compound 1 in the form of Pattern D or a pharmaceutical composition comprising Compound 1 in the form of Pattern D described herein.

Compound 1 in the form of Pattern D or a pharmaceutical composition comprising Compound 1 in the form of Pattern D described herein may also be used to treat an epileptic encephalopathy in a subject in need thereof, wherein the subject has a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBL1XR1, TCF4, TPP1, TSC1, TSC2, UBE3A, WDR45, and ZEB2.

In some embodiments, the methods described herein further comprise identifying a subject having a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBL1XR1, TCF4, TPP1, TSC1, TSC2, UBE3A, WDR45, and ZEB2 prior to administration of Compound 1 in the form of Pattern D or a pharmaceutical composition comprising Compound 1 in the form of Pattern D described herein.

### Mood Disorders

In some embodiments, the present disclosure also provides a method of treating a psychiatric disorder that comprises administering to a subject in need thereof. Compound 1 in the form of Pattern D or a pharmaceutical composition comprising Compound 1 in the form of Pattern D described herein. In some embodiments, the psychiatric disorder may be a mood disorder, for example clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. The methods comprise administering to the subject an effective amount of Compound 1 in the form of Pattern D or a pharmaceutical composition comprising Compound 1 in the form of Pattern D described herein. In some embodiments, the methods described herein provide therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD) and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case-by-case basis. Symptoms of depression, and relief of the same, may be ascertained by a physician or psychologist (e.g., by a mental state examination).

In some embodiments, the mood disorder is selected from depression, major depressive disorder, bipolar disorder, dysthymic disorder, anxiety disorders, stress, post-traumatic stress disorder, bipolar disorder, and compulsive disorders. In some embodiments, the mood disorder is major depressive disorder.

In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. The therapeutic effect can be assessed across a specified treatment period. For example, the therapeutic effect can be determined by a decrease from baseline in HAM-D total score after administering a composition described herein (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a composition described herein), is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a composition described herein is greater than or equal to 23. In some embodiments, the baseline score is at least 10, 15, or 20. In some embodiments, the HAM-D total score of the subject after treatment with a composition described herein is about 0 to 10 (e.g., less than 10; 0 to 10, 0 to 6, 0 to 4, 0 to 3, 0 to 2, or 1.8). In some embodiments, the HAM-D total score after treatment with a composition described herein is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a composition described herein. In some embodiments, the decrease in the baseline HAM-D total score to HAM-D total score after treatment with a composition described herein is at least 1, 2, 3, 4, 5, 7, 10, 25, 40, or 50). In some embodiments, the percentage decrease in the baseline HAM-D total score to HAM-D total score after treatment with a composition described herein is at least 50% (e.g., 60%, 70%, 80%, or 90%). In some embodiments, the therapeutic effect is measured as a decrease in the HAM-D total score after treatment with a composition described herein relative to the baseline HAM-D total score.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 28 days, e.g., less than or equal to 21 days or less than or equal to 14 days, since the beginning of the treatment with Compound 1 in the form of Pattern D or a pharmaceutical composition comprising Compound 1 in the form of Pattern D described herein.

In some embodiments, the therapeutic effect is a decrease from baseline in HAM-D total score after treatment with Compound 1 in the form of Pattern D or a pharmaceutical composition comprising Compound 1 in the form of Pattern D described herein. In some embodiments, the HAM-D total score of the subject before treatment with Compound 1 in the form of Pattern D or a pharmaceutical composition comprising Compound 1 in the form of Pattern D described herein is at least at least 18, at least 24 or is between 14 and 18. In some embodiments, method for treating a depressive disorder described herein results in a decrease in HAM-D total score of at least 10 or at least 15. In some embodiments, the HAM-D total score of the subject after treatment with Compound 1 in the form of Pattern D or a pharmaceutical composition comprising Compound 1 in the form of Pattern D described herein is 8 or less, e.g., 7 or less or 6 or less.

In some embodiments, the method of treating a depressive disorder provided herein provides a therapeutic effect (e.g., as measured by reduction in Clinical Global Impression-Improvement Scale (CGI)). In some embodiments, the reduction in CGI is achieved within 14, 10, 4, 3, 2, or 1 days, or 24, 20, 16, 12, 10, or 8 hours or less of starting treatment with Compound 1 in the form of Pattern D or a pharmaceutical composition comprising Compound 1 in the form of Pattern D described herein. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after start of administration of Compound 1 in the form of Pattern D or a pharmaceutical composition comprising Compound 1 in the form of Pattern D described herein).

In some embodiments, the method of treating a depressive disorder provided herein provides a therapeutic effect (e.g., as measured by a reduction in Montgomery-Asberg Depression Rating Scale (MADRS) score exhibited by the subject. For example, the method provided by the present disclosure results in a reduction of MADRS score within 14, 4, 3, 2, or 1 days; or 96, 84, 72, 60, 48, 24, 20, 16, 12, 10, 8 hours or less after the start of treatment with Compound 1 in the form of Pattern D or a pharmaceutical composition comprising Compound 1 in the form of Pattern D described herein. The MADRS is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders.

### Pain

The present disclosure also provides methods of treating pain that comprise administering to a subject in need thereof. Compound 1 in the form of Pattern D or a pharmaceutical composition comprising Compound 1 in the form of Pattern D described herein. In some embodiments, the pain comprises acute pain, chronic pain, neuropathic pain, inflammatory pain, nociceptive pain, central pain (e.g., thalamic pain), or migraine. In some embodiments, the pain comprises acute pain or chronic pain. In some embodiments, the pain comprises neuropathic pain, inflammatory pain, or nociceptive pain. In some embodiments, the pain comprises central pain (e.g., thalamic pain). In some embodiments, the pain comprises migraine.

In some embodiments, the methods provided by the present disclosure further comprise identifying a subject having pain, e.g., acute pain, chronic pain, neuropathic pain, inflammatory pain, nociceptive pain, central pain (e.g., thalamic pain), or migraine, prior to administration of Compound 1 in the form of Pattern D or a pharmaceutical composition comprising Compound 1 in the form of Pattern D described herein.

### Tremor

The present disclosure also provides method of treating tremor that comprise administering to a subject in need thereof. Compound 1 in the form of Pattern D or a pharmaceutical composition comprising Compound 1 in the form of Pattern D described herein. In some embodiments, the tremor may be cerebellar tremor or intention tremor, dystonic tremor, essential tremor, orthostatic tremor, parkinsonian tremor, physiological tremor, or rubral tremor. Tremor may include hereditary, degenerative, and idiopathic disorders such as Wilson's disease, Parkinson's disease, and essential tremor, respectively; metabolic diseases; peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (neuroleptics tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor. In some embodiments, the tremor may be familial tremor.

Tremor is an involuntary, rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke or other focal lesion disease (e.g., multiple sclerosis)) or a neurodegenerative disease.

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest or certain sensory maneuvers.

Essential tremor or benign essential tremor is the most common type of tremor. Essential tremor may be mild and non-progressive in some, and may be slowly progressive, starting on one side of the body but typically affecting both sides. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Rubral tremor is characterized by coarse slow tremor which can be present at rest, at posture, and with intention. The tremor is associated with conditions that affect the red nucleus in the midbrain, such as a stroke.

In some embodiments, the tremor is selected from essential tremor, Parkinson's tremor, or Cerebellar tremor. In some embodiments, the tremor is essential tremor.

In some embodiments, the present disclosure also provides a method of treating essential tremor that comprise administering to a subject in need thereof. Compound 1 in the form of Pattern D or a pharmaceutical composition comprising Compound 1 in the form of Pattern D described herein. In some embodiments, the method results in reduction of the essential tremor as assessed by The Essential Tremor Rating Assessment Scale (TETRAS) score. The term “The Essential Tremor Rating Assessment Scale (TETRAS)”, as used herein, refers to a scale developed to quantify severity of essential tremor and its impact on daily activities. It has an activities of daily living (ADL) section and a performance section. The ADL section has 12 items rated between 0 to 4, and the performance section has 9 items rated between 0 to 4.

In some embodiments, the reduction of the essential tremor is assessed by The Essential Tremor Rating Assessment Scale (TETRAS) upper limb score.

In some embodiments, the reduction of the essential tremor is assessed by TETRAS performance subscale score or TETRAS performance individual items.

In some embodiments, the subjects treated in accordance with the methods provided by the present disclosure have a moderate essential tremor (i.e., TETRAS score of 10-15). In some embodiments, the subjects treated herein have a TETRAS score of about 10 to about 15, or a TETRAS score of about 12, prior to treatment in accordance with the methods of the present disclosure. Treatment using Compound 1 in the form of Pattern D or a pharmaceutical composition comprising Compound 1 in the form of Pattern D described herein may reduce the TETRAS score of the subject. In some embodiments, subjects treated with Compound 1 in the form of Pattern D or a pharmaceutical composition comprising Compound 1 in the form of Pattern D described herein experience a mean reduction in TETRAS score of about 2 to 5, e.g., about 3. In some embodiments, subjects treated with Compound 1 in the form of Pattern D or a pharmaceutical composition comprising Compound 1 in the form of Pattern D described herein experience a mean reduction in TETRAS score of about 30% to about 50%, e.g., about 40%.

In some embodiments, the present disclosure also provides a method of treating essential tremor that comprise administering to a subject in need thereof. Compound 1 in the form of Pattern D or a pharmaceutical composition comprising Compound 1 in the form of Pattern D described herein. In some embodiments, the method results in reduction of the essential tremor as assessed by accelerometer-based score, e.g., accelerometer-based upper limb score. In some embodiments, the method results in reduction of the essential tremor as assessed by CGI score.

In some embodiments, the essential tremor is upper limb tremor.

In some embodiments, the subjects are selected for treatment with the compound of Compound 1 in the form of Pattern D due to a clinical diagnosis of essential tremor. In some embodiments, the subjects selected for treatment with the Compound 1 in the form of Pattern D have essential tremor, but do not have intention tremor.

The efficacy of Compound 1 in the form of Pattern D or a pharmaceutical composition comprising Compound 1 in the form of Pattern D described herein for treating essential tremor can be measured by the methods described in the following references: Ferreira, J. J. et al., “MDS Evidence-Based Review of Treatments for Essential Tremor.” Mov. Disord. 2019 July; 34 (7): 950-958; Elble, R. et al., “Task Force Report: Scales for Screening and Evaluating Tremor.” Mov. Disord. 2013 November; 28 (13): 1793-800. Deuschl G. et al. “Treatment of patients with essential tremor.” Lancet Neurol. 2011; 10:148-61. Reich S. G. et al. “Essential Tremor.” Med. Clin. N. Am. 103 (2019) 351-356. The disclosures of the references are herein incorporated in their entirety.

### Ataxia

In some embodiments, the present disclosure also provides a method of treating ataxia that comprise administering to a subject in need thereof. Compound 1 in the form of Pattern D or a pharmaceutical composition comprising Compound 1 in the form of Pattern D described herein. Ataxia, including both cerebellar ataxia and spinal ataxia (e.g., posterior spinal ataxia), generally involves the loss or failure of coordination. Patients exhibiting ataxia may have difficulty regulating the force, range, direction, velocity, and rhythm involved in posture, balance, and limb movement. Ataxia of the trunk, for example, can result in increased postural sway, and an inability to maintain the center of gravity over the base of support. Ataxia and primary or secondary symptoms of ataxic gait and tremor of the limbs may be accompanied by speech disturbance, dysphagia, abnormal ventilation and speech, and involuntary eye movements, dystonia, pyramidal or extrapyramidal symptoms, thereby substantially interfering with the activities of daily life.

As noted above, ataxia may result from a wide range of underlying diseases and conditions in a patient, including cerebellar and neurodegenerative disorders and diseases resulting from chronic or long-term exposure to toxins. Symptoms of ataxia may result from a wide range of diseases, disorders, and environmental factors, including infectious diseases, metabolic diseases, neurodegenerative diseases, genetic diseases, vascular diseases, neoplastic diseases, demyelinating diseases, neuromuscular diseases, and diseases resulting from long-term or chronic exposure to toxins (including drugs and alcohol), among a variety of others; in one embodiment, for example, the ataxia is the result of a metabolic disease, a neurodegenerative disease, a vascular disease, a neuromuscular disease, or a disease resulting from long-term or chronic exposure to toxins. Diseases, disorders, syndromes, and conditions that may result in ataxic symptoms that may be treated according to the methods described herein include, but are not limited to, amyotrophic lateral sclerosis, benign paroxysmal positional vertigo, cerebellar ataxia type 1 (autosomal recessive), cerebellar ataxias (autosomal recessive), cerebellar ataxias (dominant pure), cerebellar cortical atrophy, cerebellar degeneration (subacute), cerebellar dysfunction, cerebellar hypoplasia, cerebellar hypoplasia (endosteal sclerosis), cerebellar hypoplasia (tapetoretinal degeneration), cerebelloparenchymal autosomal recessive disorder 3, cerebelloparenchymal disorder V, cerebellum agenesis (hydrocephaly), cerebral amyloid angiopathy (familial), cerebral palsy, demyelinating disorder, dorsal column conditions, dysautonomia, dysequilibrium syndrome, dysethesis, endocrine diseases, diseases caused by chronic exposure to toxins (e.g., alcohol, drugs, antiepileptics, neuroleptics), Fragile X/Tremor ataxia syndrome, Friedreich's ataxia, frontal lobe dysfunction, genetic diseases, granulomatous angiitis of the central nervous system, Hallervorden-Spatz disease, hereditary motor and sensory neuropathy, hydrocephalus (e.g., low or normal pressure), hypotonia, congenital nystagmus, ataxia and abnormal auditory brainstem response, infantile onset spinocerebellar ataxia, Machado-Joseph disease, Meniere's disease, metabolic disorders, Miller Fisher Syndrome, Minamata disease, multiple sclerosis, muscular dystrophy, Myoclonus-ataxia, neurodegenerative diseases, olivopontocerebellar atrophy, paraneoplastic disorders, parkinsonism (atypical), peroneal muscular atrophy, phenyloin toxicity, posterior column ataxia with retinitis pigmentosa, post-polio syndrome, severe damage to the brain (caused by, e.g., head injury, brain surgery, multiple sclerosis or cerebral palsy, chronic alcohol/drug abuse, chronic exposure to toxins, viral infections, or brain tumor), spastic hemiparesis, spastic paraplegia 23, spastic paraplegia glaucoma precocious puberty, SPG, spinocerebellar ataxia, spinocerebellar ataxia (amyotrophy-deafness), spinocerebellar ataxia (dysmorphism), spinocerebellar ataxia 11, spinocerebellar ataxia 17, spinocerebellar ataxia 20, spinocerebellar ataxia 25, spinocerebellar ataxia 29, spinocerebellar ataxia 42, spinocerebellar ataxia 3, spinocerebellar ataxia (autosomal recessive 1), spinocerebellar ataxia (autosomal recessive 3), spinocerebellar ataxia (autosomal recessive 4), spinocerebellar ataxia (autosomal recessive 5), spinocerebellar ataxia (autosomal recessive, with axonal neuropathy), spinocerebellar ataxia (Machado-Joseph type II), spinocerebellar ataxia (X-linked, 2), spinocerebellar ataxia (X-linked, 3), spinocerebellar ataxia (X-linked, 4), spinocerebellar degenerescence (book type), stroke (e.g., acute or hemorrhagic), vertebral artery dissection, vertebral-basilar insufficiency, and diseases caused by vitamin deficiencies, among a variety of others. In one embodiment, the ataxia is the result of a disease selected from Spinocerebellar ataxia, Friedriech's ataxia, and fragile X/tremor ataxia syndrome. In another particular embodiment, the ataxia is the result of Spinocerebellar ataxia or fragile X/tremor ataxia syndrome.

### Tinnitus

In some embodiments, the present disclosure also provides a method of treating tinnitus that comprise administering to a subject in need thereof. Compound 1 in the form of Pattern D or a pharmaceutical composition comprising Compound 1 in the form of Pattern D described herein. Tinnitus is a condition in which those affected perceive sound in one or both ears or in the head when no external sound is present. Often referred to as “ringing” in the cars, tinnitus can occur intermittently or consistently with a perceived volume ranging from low to painfully high. However, the perceived volume of tinnitus can vary from patient to patient where an objective measure of tinnitus volume in one patient may be perceived as painful but, in another patient, the same volume may be perceived as subtle.

### Sleep Disorders

In some embodiments, the present disclosure also provides a method of treating sleep disorders that comprise administering to a subject in need thereof. Compound 1 in the form of Pattern D or a pharmaceutical composition comprising Compound 1 in the form of Pattern D described herein. For example, a sleep disorder may be a central disorder of hypersomnolence, narcolepsy type I, narcolepsy type II, idiopathic hypersomnia, Kleine-Levin syndrome, hypersomnia due to a medical disorder, hypersomnia due to a medication or substance, hypersomnia associated with a psychiatric disorder, insufficient sleep syndrome, circadian rhythm sleep-wake disorders, delayed sleep-wake phase disorder, advanced sleep-wake phase disorder, irregular sleep-wake rhythm, non-24-hour sleep-wake rhythm disorder, shift work disorder, jet lag disorder, circadian rhythm sleep-wake disorder not otherwise specified (NOS).

### Combination Therapy

In some embodiments, Compound 1 in the form of Pattern D or a pharmaceutical composition comprising Compound 1 in the form of Pattern D described herein may be administered in combination with another agent or therapy. A subject to be administered. In some embodiments, the present disclosure also provides a method of treating ataxia that comprise administering to a subject in need thereof. Compound 1 in the form of Pattern D or a pharmaceutical composition comprising Compound 1 in the form of Pattern D described herein may have a disease, disorder, or condition, or a symptom thereof, that would benefit from treatment with another agent or therapy. These diseases or conditions can relate to epilepsy or an epilepsy syndrome (e.g., absence seizures, juvenile myoclonic epilepsy, or a genetic epilepsy) or tremor (e.g., essential tremor).

### Antiepilepsy Agents

Anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide.

### Analgesics

Analgesics are therapeutic agents that are used to relieve pain. Examples of analgesics include opiates and morphinomimetics, such as fentanyl and morphine; paracetamol; NSAIDs, and COX-2 inhibitors. Given the ability of Compound 1 to treat pain via inhibition of T-type calcium channels (e.g., Cav3.1, Cav3.2, and Cav3.3), combination with analgesics is particularly envisioned.

### Tremor Medications

Tremor medications include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide.

### EXEMPLIFICATION OF THE INVENTION

### Example 1. Preparation of a New Crystalline Form of Compound 1 (Pattern D)

The purpose of this study was to conduct a polymorph investigation on Compound 1.

The starting material, Compound 1, was characterized by X-Ray Powder Diffraction (XRPD), Polarized Light Microscopy (PLM), Thermo-Gravimetric Analysis (TGA) and Differential Scanning calorimetry (DSC). The characterization results indicated that the starting material is an anhydrous crystalline solid. DSC showed a singular endotherm with an onset of 224.5° C. The crystalline type in the starting material was determined to be the previously characterized Pattern C, as described, e.g., in WO/2021/007487, the entire contents of which are hereby incorporated herein by reference.

Amorphous material was generated through ball milling in the experiments with the goal to obtain a new crystalline form. Those experiments led to discovery of a new crystalline form labeled Pattern D. Additional experiments were set up starting with crystalline Pattern C, which showed that Pattern D crystallizes at water activities of 0.8 and above. Pattern D was determined to be a channel trihydrate, the form identification was confirmed by Single Crystal XRD. No form conversion was observed upon heating Pattern D to 90° C., however slight peak broadening was observed. When a sample containing Pattern D was further heated to 150° C., it turned amorphous.

Starting with amorphous ball milled material, polymorph screening was performed under four different experimental conditions and resulted in formation of Pattern D. Table 1 and Table 2 below show experimental conditions used in the experiments and the resulting crystalline form obtained.

TABLE 1
Polymorph Screening Experiments from Amorphous API.

| | | Pattern |
|---|---|---|
| Experiment | Solvents | (as determined by XRPD) |
| Evaporation | EtOAc | Pattern C |
| Slurry | EtOAc | Pattern C |
| Temperature Cycling | H₂O | Pattern D |
| Temperature Cycling | Acetone/H₂O (1:1) | Gel → Pattern D* |

*Crystalline material obtained in gel after vial left to sit for 1 week as a large block-like crystal.

TABLE 2
Water Activity Experiments from Amorphous API.

| Solvents | Pattern (as determined by XRPD) |
|---|---|
| Acetone/H₂O | Pattern C |
| (a_{w} = 0.2, 941/59) | |
| Acetone/H₂O | Pattern C |
| (a_{w} = 0.4, 857/143) | |
| Acetone/H₂O | Pattern C |
| (a_{w} = 0.6, 726/274) | |
| Acetone/H₂O | Pattern D |
| (a_{w} = 0.8, 492/508) | |
| H₂O | Pattern D |

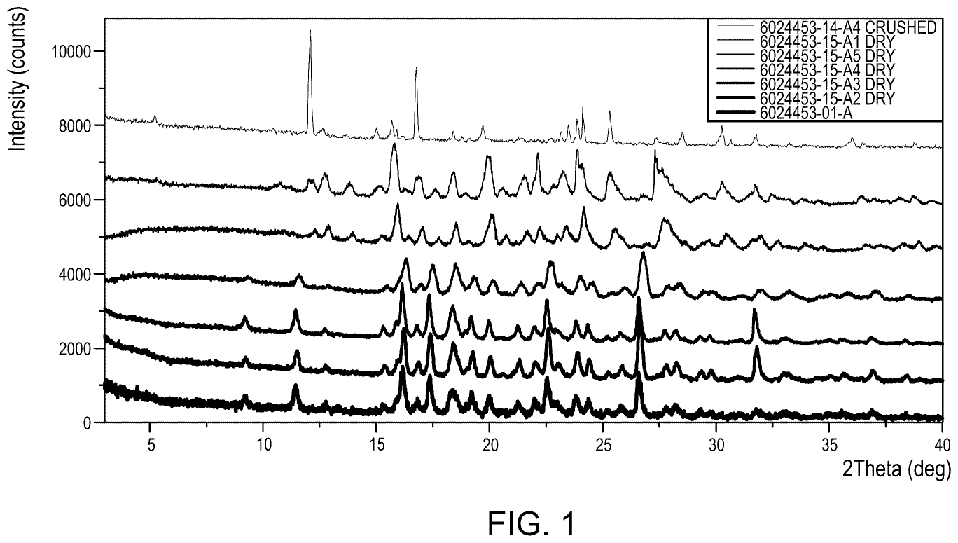

FIG. 1 is an is an X-ray powder diffraction (XRPD) spectrum of water activity experiments comparing Pattern C and Pattern D.

### Example 2. Characterization of a New Crystalline Form of Compound 1 (Pattern D)

Pattern D was observed in many conditions at a water activity of 0.8 and higher. Pattern D was characterized by various techniques.

FIG. 2 shows an XRPD spectrum of Pattern D. Table 3 below also shows the list of peaks observed in the XRPD spectrum of Pattern D.

TABLE 3
XRPD peak list for Pattern D.

| | | FWHM Left | | Rel. |
|---|---|---|---|---|
| Pos. (° 2θ) | Height (cts] | (° 2θ) | d-spacing (Å) | Int. (%) |
| 5.230397 | 763.954500 | 0.051168 | 16.89615 | 8.51 |
| 10.420430 | 325.714200 | 0.102336 | 8.48955 | 3.63 |
| 10.647290 | 383.653600 | 0.063960 | 8.30917 | 4.27 |
| 11.045250 | 247.374400 | 0.102336 | 8.01067 | 2.75 |
| 11.981570 | 4109.730000 | 0.063960 | 7.38668 | 45.76 |
| 12.603310 | 1422.221000 | 0.089544 | 7.02365 | 15.84 |
| 13.628080 | 588.600700 | 0.089544 | 6.49772 | 6.55 |
| 14.876240 | 588.302900 | 0.089544 | 5.95523 | 6.55 |
| 15.064850 | 447.714500 | 0.063960 | 5.88110 | 4.99 |
| 15.598220 | 3276.813000 | 0.076752 | 5.68118 | 36.49 |
| 16.111860 | 560.822100 | 0.115128 | 5.50120 | 6.24 |
| 16.701260 | 6162.353000 | 0.089544 | 5.30837 | 68.62 |
| 17.425680 | 512.199900 | 0.102336 | 5.08929 | 5.70 |
| 18.175510 | 1083.506000 | 0.089544 | 4.88098 | 12.06 |
| 18.737560 | 371.310100 | 0.127920 | 4.73582 | 4.13 |
| 19.814360 | 2081.657000 | 0.089544 | 4.48082 | 23.18 |
| 20.381210 | 1083.810000 | 0.076752 | 4.35746 | 12.07 |
| 20.725690 | 454.841700 | 0.127920 | 4.28581 | 5.06 |
| 21.245970 | 2196.599000 | 0.063960 | 4.18202 | 24.46 |
| 21.401240 | 1597.410000 | 0.063960 | 4.15203 | 17.79 |
| 21.865380 | 1331.243000 | 0.076752 | 4.06493 | 14.82 |
| 22.642500 | 620.053300 | 0.089544 | 3.92714 | 6.90 |
| 23.054720 | 1675.199000 | 0.115128 | 3.85785 | 18.65 |
| 23.809610 | 6912.878000 | 0.063960 | 3.73722 | 76.97 |
| 24.069630 | 1881.316000 | 0.089544 | 3.69743 | 20.95 |
| 25.177120 | 6623.757000 | 0.063960 | 3.53725 | 73.75 |
| 25.534480 | 438.613500 | 0.127920 | 3.48855 | 4.88 |
| 27.283150 | 8980.884000 | 0.063960 | 3.26880 | 100.00 |
| 27.958260 | 327.082200 | 0.127920 | 3.19138 | 3.64 |
| 30.209570 | 2845.963000 | 0.063960 | 2.95848 | 31.69 |
| 30.534000 | 224.026900 | 0.127920 | 2.92779 | 2.49 |
| 32.369990 | 1655.268000 | 0.063960 | 2.76580 | 18.43 |
| 32.762050 | 423.087800 | 0.179088 | 2.73359 | 4.71 |
| 36.277670 | 1191.883000 | 0.102336 | 2.47634 | 13.27 |
| 36.877090 | 430.302600 | 0.102336 | 2.43746 | 4.79 |
| 37.786860 | 312.149400 | 0.127920 | 2.38084 | 3.48 |
| 38.627630 | 787.111000 | 0.115128 | 2.33093 | 8.76 |

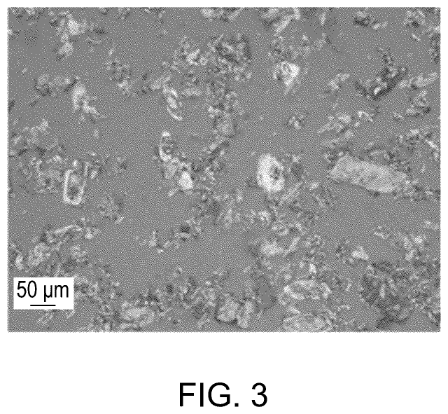

FIG. 3 is a polarized light microscopy (PLM) image of Pattern D. FIG. 3 shows that Pattern D crystalizes as block-like crystals.

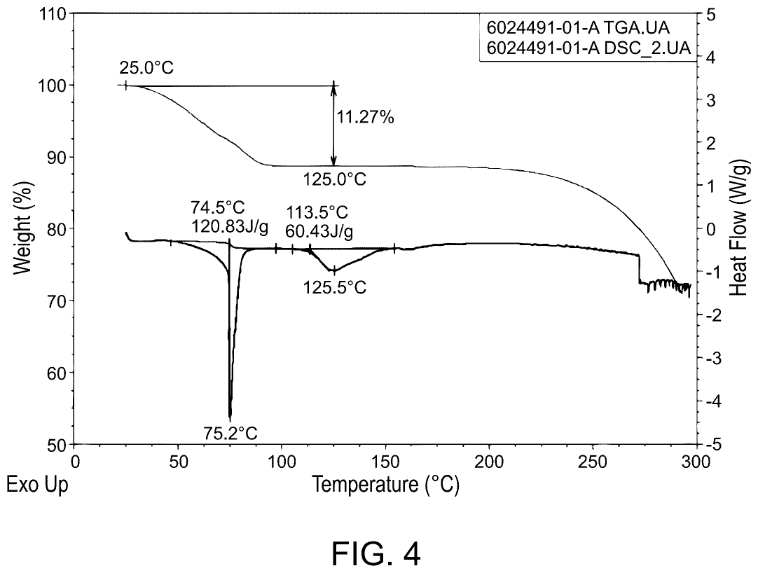

FIG. 4 shows the results of a thermo-gravimetric analysis (TGA) and differential scanning calorimetry (DSC) analysis of Pattern D. FIG. 4 shows a 11.27% weight loss to 125° C. in the TGA (˜3.09:1 H₂O:API) and two broad endotherms with onsets at 74.5° C. and 113.5° C. in the DSC curve. KF showed 11.1% water content (˜3:1 H₂O:API).

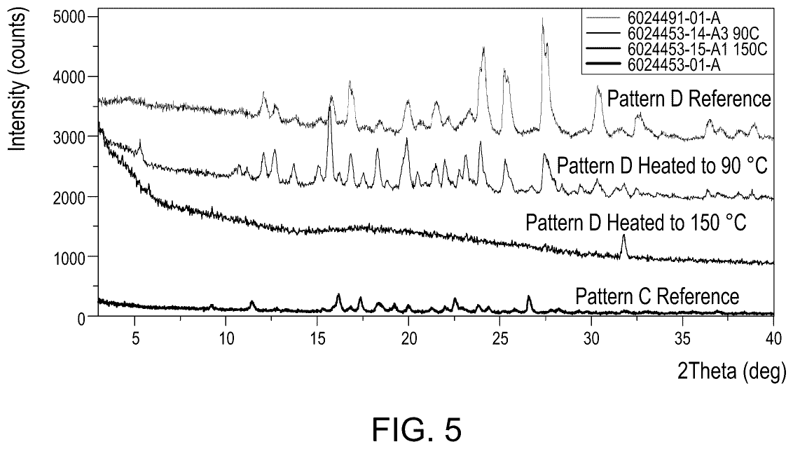

FIG. 5 is an overlay of XRPD spectra of Pattern D heated to 90° C. and cooled back to room temperature; Pattern D heated to 150° C. and cooled back to room temperature; as well as Pattern C and Pattern D reference spectra. FIG. 5 indicates that when Pattern D is heated to 90° C., there is significant peak broadening, yet there is no form conversion. When Pattern D is heated further to 150° C., the sample turns amorphous.

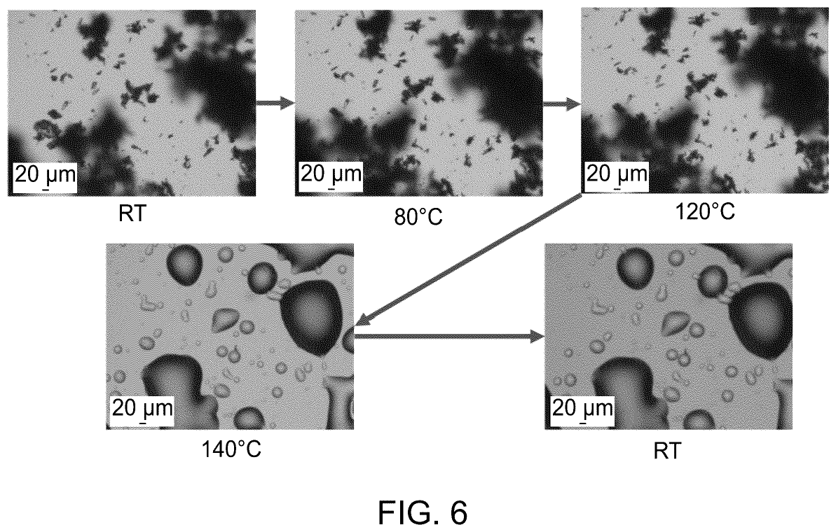

FIG. 6 is an image of Pattern D heated to different temperatures obtained using hot stage microscopy (HSM). FIG. 6 confirms the results shown in FIG. 5.

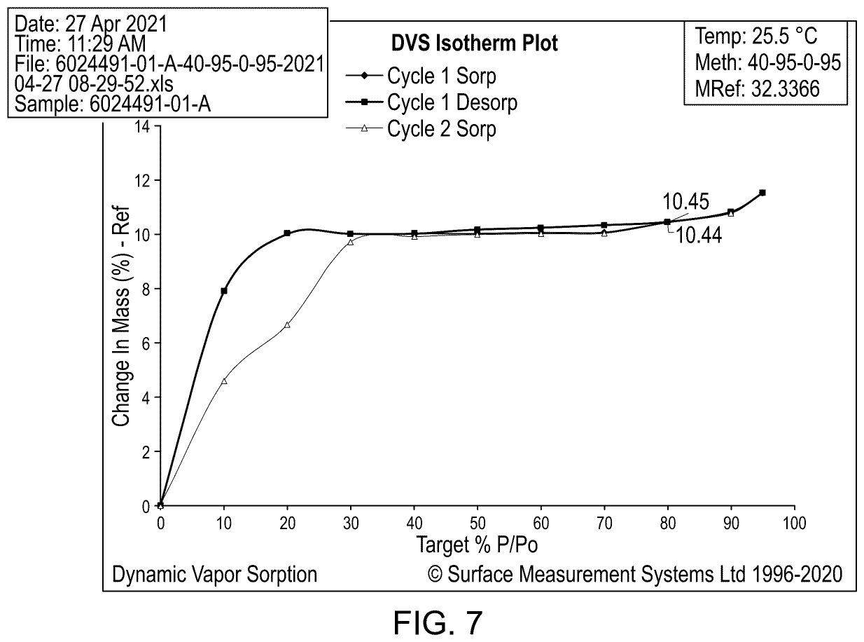

FIG. 7 is a Dynamic Vapor Sorption (DVS) spectrum collected on Pattern D. FIG. 7 shows that the material gained ˜10.45% mass at 80% RH. The hydrate was desolvated and lost the adsorbed moisture at RH close to 0, and re-adsorbed approximately 10.5% water molecules by RH of 20%.

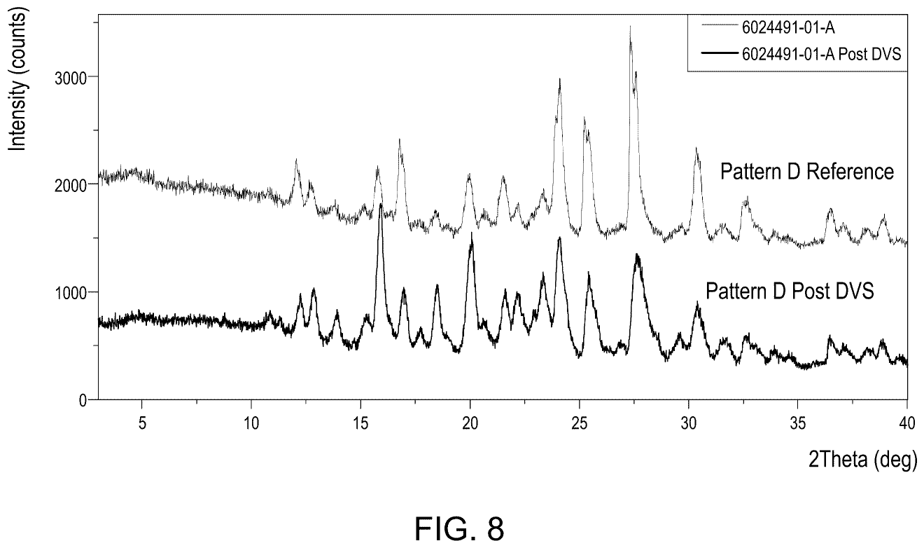

FIG. 8 is an XRPD spectrum taken on Pattern D post DVS, which shows no clear form change.

### Water Solubility of Pattern D

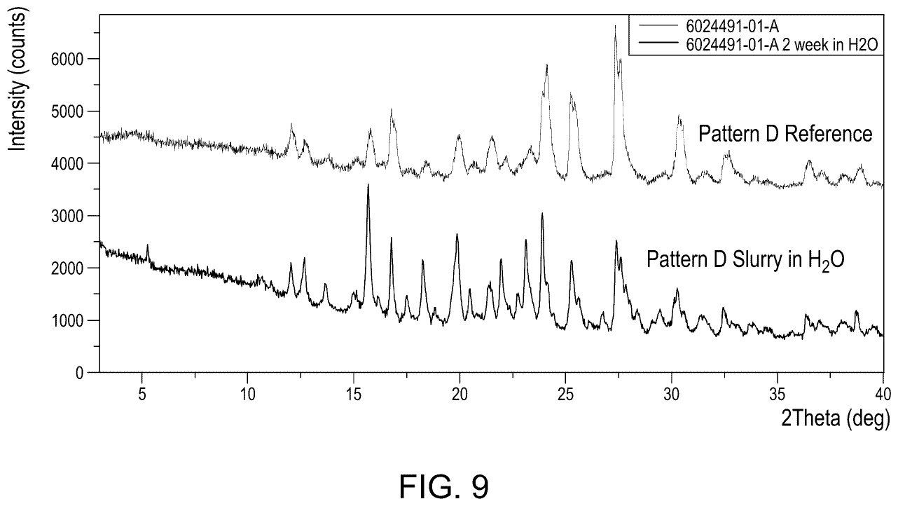

Water solubility of Pattern D was measured by HPLC to determine how much the water solubility drops between Pattern C and Pattern D. To measure the solubility of Pattern D in H₂O, ˜150 mg of Pattern D was slurried for 2 weeks at RT in 1 mL of H₂O. Pattern D was found to have a solubility of 77.24 mg/mL in H₂O, compared to solubility of greater than 154 mg/mL observed for Pattern C. FIG. 9 is an overlay of XRPD spectra of a 2-week slurry of Pattern D in H₂O and of Pattern D reference and shows no form conversion.

### Example 3. Crystal Structure of Pattern D

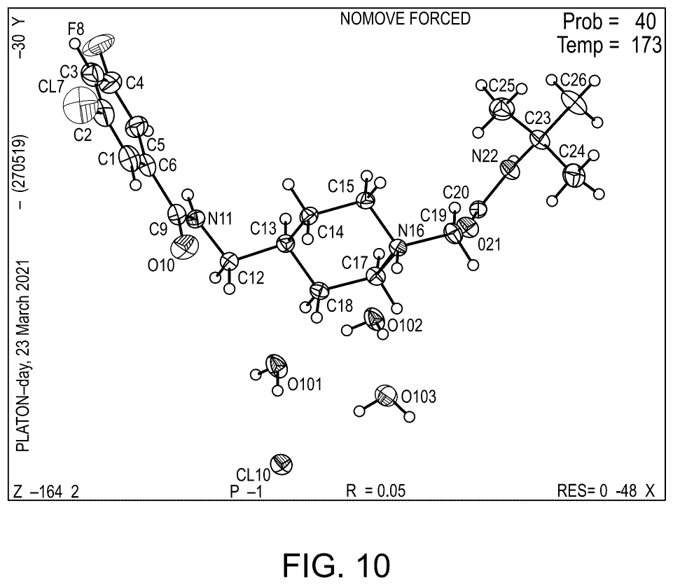

The proposed structure of Form D was verified by single crystal x-ray structure solution. FIG. 10 is a Thermal Ellipsoid plot of the compound in the crystal of Pattern D. A plate-like single crystal with high diffraction quality was immersed in Paratone-N (an oil based cryoprotectant). The crystal was mounted on a mylar loop in a random orientation and immersed in a stream of liquid nitrogen at 173K. The X-ray intensity data were measured on a Bruker D8 VENTURE (IμS microfocus X-ray source, Cu Kα, λ=1.54178 Å, PHOTON CMOS detector) diffractometer. The frames were integrated with the Bruker SAINT software package. The integration of the data using a triclinic unit cell yielded a total of 73495 reflections to a maximum θ angle of 75.070 (0.83 Å resolution), of which 4988 were independent (completeness=99.9%, Rᵢₙₜ=3.39%) and were greater than 2σ(F²). The final cell constants of a=8.584(3) Å, b=9.11(4) Å, c=17.043 Å, α=81.24(3)°, β=88.90(3)°, γ=67.176(16)°, cell volume=1213.0(10) Å³, are based upon the refinement of the XYZ-centroids of 4988 reflections above 20 σ(I) with 2.626°<θ<75.070°. Data were corrected for absorption effects using the Multi-Scan method (SADABS). The absorption coefficient μ of this material is 2.761 mm⁻¹ at this wavelength (1.54178 Å). The calculated minimum and maximum transmission coefficients (based on crystal size) are 0.6649 and 0.7538. The agreement factor for the averaging was 4.74% based on intensity.

The structure was solved with the ShelXTL structure solution program using Intrinsic Phasing and refined with ShelXL (Sheldrick, G. M. SHELXTL-2014, Crystallographic Computing System; Bruker Analytical X-Ray Instruments: Madison, WI, 2014; (b) Sheldrick, G. M. A Short History of SHELX, Acta Cryst. 2008, A64, 112), (Version 2014/7) refinement package using full-matrix least-squares on F² contained in ShelXL (Sheldrick, G. M. SHELXTL-2014, Crystallographic Computing System; Bruker Analytical X-Ray Instruments: Madison, WI, 2014; (b) Sheldrick, G. M. A Short History of SHELX, Acta Cryst. 2008, A64, 112; Dolomanov, O. V., Bourhis, L. J., Gildea, R. J, Howard, J. A. K. & Puschmann, H. (2009), J. Appl. Cryst. 42, 339-341) using the space group P1̅, with Z=2 for the formula unit, C₁₉H₂₇ClFN₃O₂·HCl, 3(H₂O). All non-hydrogen atoms were refined anisotropically. The positions of the hydrogen atoms connected to carbon atoms were calculated geometrically and refined using the riding model, but the hydrogen atoms connected to the nitrogen N13 and the solvent water molecules were located in the difference Fourier map and was allowed to refine freely to its final position. The final anisotropic full-matrix least-squares refinement on F² with 299 variables converged at R₁=4.74%, for the observed data and wR₂=13.28% for all data. The goodness-of-fit was 1.092. The largest peak in the final difference electron density synthesis was 0.660 e⁻/Å³ and the largest hole was −0.910 e⁻/Å³. Based on the final model, the calculated density is 1.299 g/cm³ and F (000), 504 e⁻. The contents of the asymmetric unit of the crystal structure and its thermal ellipsoid representation are shown in FIG. 10.

### Structural Details and Interpretation

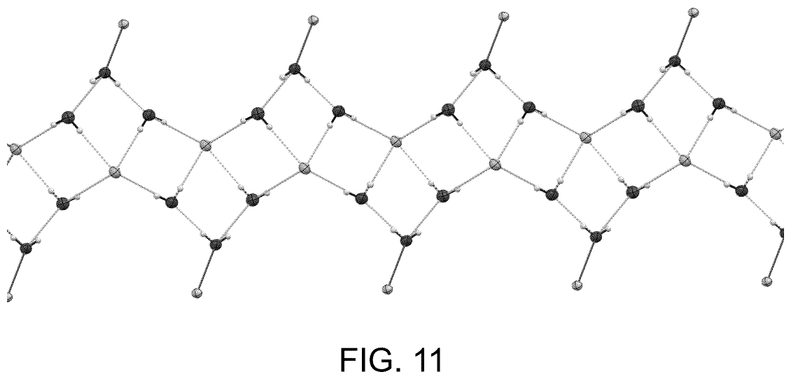

The structure of Compound 1 was given as a hydrochloride salt obtained from an acetone/water solvent system. Three water molecules were identified and allowed to refine their occupancy freely, but converged to full occupancy, indicating a highly ordered structure formed by the water molecules. FIG. 11 shows hydrogen bonding network in Pattern D formed by the water and chloride anions. The API molecules are connected by the displayed nitrogen atoms on the periphery. This is further emphasized with the position of the hydrogen atoms observed in the Fourier peaks and assigned as real atoms rather than geometrically idealized locations.

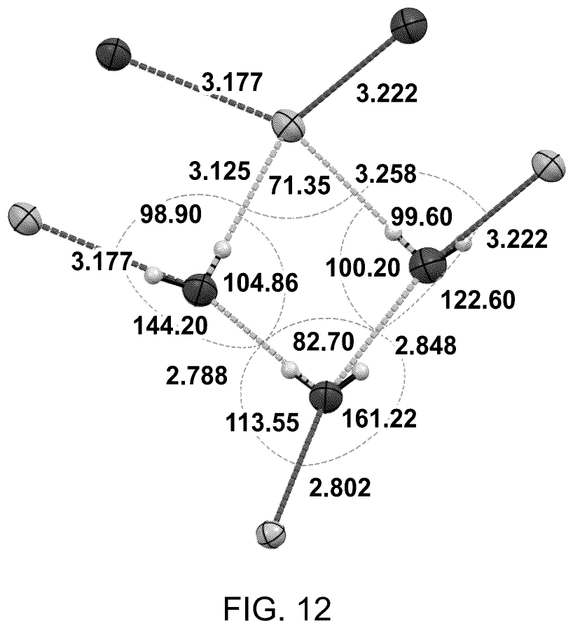

This level of order within a solvent network is quite unusual. In addition, the distances and angles within the hydrogen-bonding network are close to idealized as shown in FIG. 12. Specifically, FIG. 12 shows distances (Angstroms, red) and Angles (degrees, blue) in the solvent-anion network. Displayed are only the three solvent molecules and one anion per API unit, and the atoms with which they interact. The donor-acceptor distance of 2.8 Angstroms indicates a moderate hydrogen bond between the water molecules, and the gap between the water-water hydrogen-bonds and the water-chloride hydrogen bonds corresponds with the difference in the atomic radii between oxygen and chlorine (˜0.3 Angstroms). In addition, a Fourier peak was located on the tertiary amine N16 that corresponded to a hydrogen, with a contact distance inferring the presence of a hydrogen bond with 0102 in the solvent water. Since the hydrogen atoms in that water molecule had been located and assigned directly, this indicated protonation of the amine, and has been refined accordingly.

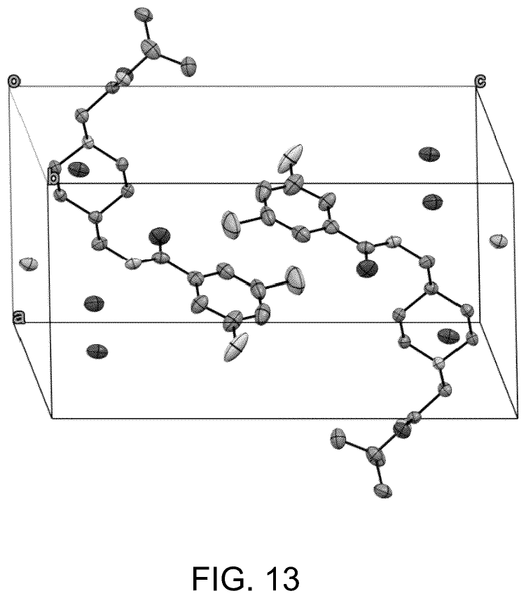

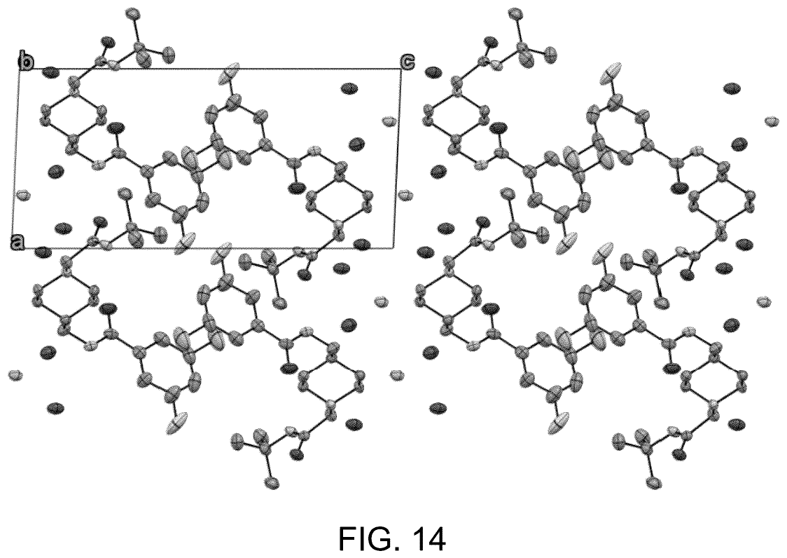

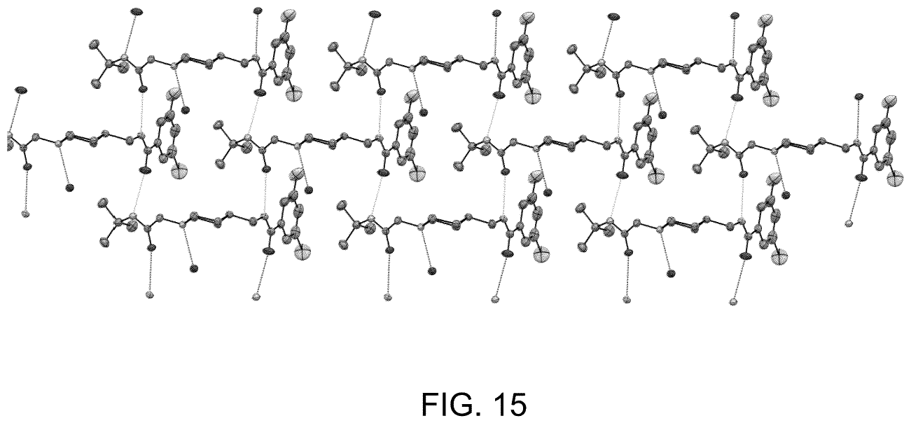

The packing of Compound 1 is also of interest. With the hydrogen bonding network in place, the solvents are extremely stable, but also still exist in channels, as shown in FIGS. 13 and 14. Specifically, FIG. 13 shows a unit cell of Compound 1, with hydrogen atoms omitted for clarity, while FIG. 14 shows packing of Compound 1 as viewed along the b-axis. FIG. 15 shows hydrogen bonding between molecules of Compound 1, with hydrogen atoms omitted for clarity. FIG. 15 shows that the API molecules themselves also have a separate network of short contacts between amide groups (2.899 and 2.922 Angstrom contact distances), which organize the API molecules into two-dimensional sheets. These sheets are then paired by slipped π stacking interactions on the di-substituted phenyl rings. The solvent channels exist in sheets between these double-sheet formations and are what connect them together.

The chloride anion within the solvent channels is bound only to water molecules—there are no direct API-anion contacts in this configuration. It is quite possible that the water molecules in these channels have multiple potential hydrogen-bonding motifs depending upon the quantity of water available. In addition, the stability of the chloride anion may be dependent upon maintaining some degree of solvation. Though Compound 1 crystallized as a tri-hydrate, it is quite likely that these water molecules do not have the same degree of mobility within the channel, and their lability should be considered separate and iterative. In the event of complete dehydration, the channel will be occupied exclusively by unbound and exposed chloride anions. Multiple potential pathways may be envisioned for such system to stabilize, such as the deprotonation of the API followed by the loss of the unbound HCl gas. More likely, the channel will repopulate with some degree of hydration quite rapidly upon exposure to air. In short, this crystal forms as a tri-hydrate with solvent channels but may behave as a mono or di-hydrate to retain the level of hydration necessary to stabilize the chloride anion.

### EQUIVALENTS

Those skilled in the art will recognize, or be able to ascertain using no more than routine experimentation, many equivalents to the specific embodiments and methods described herein. Such equivalents are intended to be encompassed by the scope of the present invention. All patents, patent applications, and literature references cited herein are hereby expressly incorporated by reference.

What is claimed is:

1. A crystalline form of N-((1-(2-(tert-butylamino)-2-oxoethyl)piperidin-4-yl)methyl)-3-chloro-5-fluorobenzamide hydrochloride salt trihydrate (Compound 1) characterized by an x-ray powder diffraction (XRPD) pattern comprising at least one peak at the diffraction angle (° 2θ) selected from the group consisting of:
a peak at approximately 12.0°;
a peak at approximately 15.6°;
a peak at approximately 16.7°;
a peak at approximately 19.8°;
a peak at approximately 21.2°;
a peak at approximately 24.1°;
a peak at approximately 25.2°;
a peak at approximately 27.3°; and
a peak at approximately 30.2°.

2. The crystalline form of claim 1 characterized by an XRPD pattern comprising the following peaks at the diffraction angle (° 2θ):
a peak at approximately 25.2°; and
a peak at approximately 27.3°.

3. The crystalline form of claim 1 characterized by an XRPD pattern comprising the following peaks at the diffraction angle (° 2θ):
a peak at approximately 24.1°;
a peak at approximately 25.2°; and
a peak at approximately 27.3°.

4. The crystalline form of claim 1 characterized by an XRPD pattern comprising the following peaks at the diffraction angle (° 2θ):
a peak at approximately 16.7°;
a peak at approximately 24.1°;
a peak at approximately 25.2°; and
a peak at approximately 27.3°.

5. The crystalline form of claim 1 characterized by an XRPD pattern comprising the following peaks at the diffraction angle (° 2θ):
a peak at approximately 12.0°;
a peak at approximately 16.7°;
a peak at approximately 24.1°;
a peak at approximately 25.2°; and
a peak at approximately 27.3°.

6. The crystalline form of claim 1 characterized by an XRPD pattern substantially the same as the XRPD pattern depicted in FIG. 2.

7. The crystalline form of any one of claims 1-6, wherein the crystalline form has a melting point onset as determined by differential scanning calorimetry (DSC) at about 75.2° C.

8. The crystalline form of any one of claims 1-7, wherein the crystalline form has a crystal structure characterized as a space group P1̅.

9. The crystalline form of claim 8, wherein the crystal structure is characterized by an asymmetric unit cell with a volume of 1213.0(10) Å³ and 3-D parameters of a=8.584(3) Å; b=9.11(4) Å; c=17.043 Å.

10. A pharmaceutical composition comprising the crystalline form of any one of claims 1-8 and a pharmaceutically acceptable carrier.

11. The pharmaceutical composition of claim 10, further comprising a modified release-polymer.

12. The pharmaceutical composition of claim 11, wherein the modified-release polymer is selected from the group consisting of a hydrophilic matrix polymer, a hydrophobic matrix polymer and a polyacrylate polymer.

13. The pharmaceutical composition of claim 12, wherein the hydrophilic matrix polymer is hypromellose.

14. The pharmaceutical composition of any one of claims 10-13, wherein the pharmaceutical composition is for oral administration.

15. A method of treating a neurological disorder comprising administering to a subject in need thereof the crystalline form of any one of claims 1-9 or the pharmaceutical composition of any one of claims 10-14.

16. The method of claim 15, wherein the neurological disorder is tremor.

17. The method of claim 16, wherein the tremor is essential tremor, Parkinson's tremor, cerebellar tremor or CACNA1G tremor.

18. The method of claim 17, wherein the tremor is essential tremor.

19. A method of treating a psychiatric disorder in a subject in need thereof, said method comprising administering to said subject the crystalline form of any one of claims 1-9 or the pharmaceutical composition of any one of claims 10-14.
